(19) United States
(12) Patent Application Publication
(10) Pub. No.: US 2010/0261796 A1
(43) Pub. Date: Oct. 14, 2010

(54) Use of Novel Compounds for IBD Treatment

(75) Inventors: Pierre J-M Rivière, San Diego, CA (US); Chia-Ping Chang, San Diego, CA (US); Pierre Desreumaux, Lille (FR)

Correspondence Address: FOLEY AND LARDNER LLP;SUITE 500, 3000 K STREET NW, WASHINGTON, DC, 20007, US

(73) Assignee: Ferring B.V., Hoofddorp (NL)

(21) Appl. No.: 11/919,188

(22) PCT Filed: Apr. 26, 2006
(86) PCT No.: PCT/US06/15624

§ 371 Date: Jun. 22, 2010

Related U.S. Application Data
(60) Provisional application No. 60/675,972, filed on Apr. 28, 2005.

(51) Int. Cl. A61K 31/17; A61P 1/00

(52) U.S. Cl. 514/596

(57) ABSTRACT

The present invention relates to use of novel compounds for the manufacture of a medicament for treatment of inflammatory bowel disease (IBD) as well as to a method for treatment of IBD, wherein said compounds are administered. The compounds are represented by the general formula (I), as further defined in the specification.

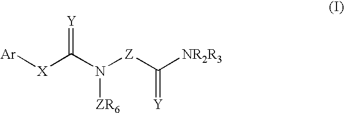

### FIELD OF THE INVENTION

The present invention relates to use of novel compounds for the manufacture of a medicament for treatment of inflammatory bowel disease (IBD) as well as to a method for treatment of IBD, wherein said compounds are administered.

### BACKGROUND

Inflammatory bowel disease, also known as IBD, is a well known disorder. It includes Crohn's disease and ulcerative colitis. Although the latter are two distinct conditions, they share the common feature of inflammation across the wall of the gastrointestinal tract. See e.g. Hendrickson, B. A. et al. in Clinical Microbiology Reviews Jan. pp 79-94 (2002) for a review of these conditions. Approved therapies for IBD are limited to mesalazine (e.g. Pentasa®), steroids (e.g. budesonide), and the more recently approved anti-TNF modulators (e.g. infliximab, Remicade®). Since far from all patients experience adequate relief of symptoms with the existing drugs, there is still a need for new therapeutics to treat IBD.

The PCT application published as WO 02/085866 discloses compounds active as CB2 agonists and their use in the management of pain.

The PCT application published as WO 2004/085385 discloses compounds that may have effect in the treatment of IBD.

### DISCLOSURE OF THE INVENTION

The compounds disclosed herein have been found to exhibit surprisingly potent properties in the treatment of IBD. More specifically, the present invention relates to the use of a compound having the general formula (I):

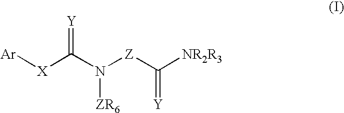

wherein
X is selected from the radicals —NR₁— and —CHR₁—;
Y is independently selected from O and S;
Z is independently selected from a C₁₋₇ straight or C₄₋₈ branched alkylene chain, a C₂₋₇ alkenylene chain and a part of a C₃₋₈ cycloalkyl or C₅₋₈ cykloalkenyl ring structure;
Ar is an aryl group selected from aromatic carbocyclic ring systems, five- or six-membered heteroaromatic ring systems and bicyclic heteroaromatic ring systems;
R₁, R₂ and R₃ are independently selected from a group of substituents (a)-(d) consisting of:
(a) H;
(b) C₁₋₆ straight or C₄₋₈ branched chain alkyl;
(c) C₃₋₈ cycloalkyl or C₅₋₈ cykloalkenyl; and
(d) C₂₋₆ alkenyl or alkynyl;
   wherein the substituents (b)-(d) optionally have at least one substituent independently selected from a group (e)-(i) consisting of:
(e) Ar, O—Ar or S—Ar;
(f) OH, O-alkyl or S-alkyl, where alkyl is selected from the substituents (b)-(c);
(g) NR₄R₅, where R₄ and R₅ are independently selected from the substituents (a)-(d) or optionally together form a nitrogen containing ring structure comprising from 2 to 5 carbon atoms;
(h) NH—C(O)-alkyl, C(O)-alkyl, O—C(O)-alkyl or S—C(O)-alkyl, where alkyl is selected from the substituents (b)-(c); and
(i) F, Cl or Br;
   R₆ is selected from a group consisting of Ar and the substituents (a)-(c), where (b) and (c) are optionally substituted with at least one of the substituents (e)-(i);
   Ar optionally has at least one substituent independently selected from the substituents (b)-(i); and
   tautomers, solvates, and pharmaceutically acceptable salts of said compound;
   for the manufacture of a medicament for treatment of inflammatory bowel disease (IBD). Treatment of Crohn's disease and ulcerative colitis, alone or in combination, is especially preferred.

For the purposes of the present invention, the following terminology is used.

Aromatic carbocyclic ring systems includes phenyl and naphthyl.

A five-membered heteroaromatic ring system is a monocyclic aromatic ring system having five ring atoms, wherein 1, 2 or 3 ring atoms are independently selected from N, O and S. Preferred such ring systems are selected from a group consisting of thienyl, furyl, pyrrolyl, imidazolyl, thiazolyl, oxazolyl, pyrazolyl, isothiazolyl, isoxazolyl and tetrazolyl.

A six-membered heteroaromatic ring system is a monocyclic aromatic ring system having six ring atoms, wherein 1, 2 or 3 ring atoms are independently selected from N, O and S. It is preferably selected from a group consisting of pyridyl, pyrazinyl, pyrimidinyl, triazinyl and pyridazinyl.

A bicyclic heteroaromatic ring system is a ring system having two five- or six-membered heteroaromatic rings, or a phenyl and a five- or six-membered heteroaromatic ring, or a phenyl and a heterocyclyl ring, or a five- or six-membered heteroaromatic ring and a heterocyclyl ring; connected by a ring fusion, said bicyclic heteroaromatic ring system comprising 8 to 12 ring atoms, wherein 1, 2 or 3 of the ring atoms are independently selected from N, O and S. It is preferably selected from a group consisting of indole, quinoline, tetrahydroquinoline, isoquinoline, tetrahydroisoquinoline, 1,4-benzodioxan, coumarin, benzofuran, 1,2-benzisoxazole, benzothiophene, benzoxazole, benzthiazole, benzimidazole, benztriazole, pyrolizidine and quinolizidine.

A heterocyclyl or heterocyclic moiety is a saturated or partially saturated ring system having 3 to 7 ring atoms, wherein 1, 2 or 3 ring atoms are independently selected from N, O and S. Heterocyclyl moieties are preferably selected from a group consisting of aziridine, oxirane, thiirane, azetidine, oxetane, thietane, pyrrolidine, pyrroline, imidazolidine, pyrazolidine, dioxolane, tetrahydrofuranyl, piperidine, piperazine, morpholine, tetrahydropyranyl, 1,4-dioxanyl, homopiperidinyl, homopiperazinyl and hexamethylene oxide.

It deserves mentioning that e.g. also isopropyl and 2-n-butyl groups are encompassed by the expression C₁₋₆ straight chain alkyl, as said expression is not related to the binding site of the straight chain in question.

C₁₋₆ denotes having from one to six carbon atoms, including any number therebetween, and this nomenclature is used analogously herein.

Examples of pharmaceutically acceptable salts comprise acid addition salts, e.g. a salt formed by reaction with hydrohalogen acids, such as hydrochloric acid, and mineral acids, such as sulphuric acid, phosphoric acid and nitric acid, as well as aliphatic, alicyclic, aromatic or heterocyclic sulphonic or carboxylic acids, such as formic acid, acetic acid, propionic acid, succinic acid, glycolic acid, lactic acid, malic acid, tartaric acid, citric acid, ascorbic acid, maleic acid, hydroxymaleic acid, pyruvic acid, p-hydroxybenzoic acid, embonic acid, methanesulphonic acid, ethanesulphonic acid, hydroxyethanesulphonic acid, halobenzenesulphonic acid, toluenesulphonic acid and naphtalenesulphonic acid.

A compound (I) wherein said X is a radical —NR₁— is preferred. It is particularly preferred that R₁ is H.

It is moreover preferred that said Y in the formula (I) represents O, i.e. an oxygen atom.

The group Ar is preferably selected from phenyl and naphthyl. The naphthyl group may be either a 1- or 2-naphthyl group.

Said Z is preferably selected from —CH₂—, —(CH₂)₂—, —(CH₂)₃—, —(CH₂)₅—, —(CH₂)₆—, —(CH₂)₇— and trans-2-cyclohexylene.

Said R₆ is preferably selected from isopropyl, cyclopentyl, cyclohexyl, phenyl, 4-n-butylphenyl, 4-isopropylphenyl and 2-naphthyl.

It is preferred that said R₂ and R₃ are independently selected from H and 4-chlorobenzyl.

In the most preferred embodiment, said compound having the formula (I) is selected from a group consisting of:
4-[3-phenyl-1-(6-phenylhexyl)ureido]butyramide (2);
4-[1-(4-butylbenzyl)-3-phenylureido]butyramide (3);
4-[1-(4-isopropylbenzyl)-3-phenylureido]butyramide (4);
4-[(1-(4-methylpentyl)-3-phenylureido]butyramide (5);
N-(4-chlorobenzyl)-4-[1-(3-cyclohexylpropyl)-3-phenylureido]butyramide (6);
trans-2-[1-(3-cyclohexylpropyl)-3-phenylureido]cyclohexanecarboxamide (7);
4-[1-(3-cyclohexylpropyl)-3-naphthalen-2-yl-ureido]-butyramide (10);
4-[1-(2-naphthalen-2-yl-ethyl)-3-phenylureido]butyramide (11);
4-[1-(2-cyclohexylethyl)-3-phenylureido]butyramide (12);
4-(1-phenethyl-3-phenylureido)butyramide (13);
4-(1-benzyl-3-phenylureido)butyramide (14);
4-[1-(3-cyclopentylpropyl)-3-phenylureido]butyramide (15);
4-[3-phenyl-1-(5-phenylpentyl)ureido]butyramide (16); and
4-[1-(3-cyclohexylpropyl)-3-phenylureido]butyramide (17).

The number in parenthesis denotes the compound as referred to in the following.

Compound 17 is the very most preferred embodiment of the use of the present invention. Its structure is provided below:

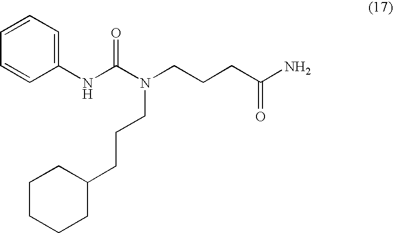

A second aspect of the present invention relates to the use of a compound with the formula (I), wherein X is a radical —CHR₁—. It is preferred that said radical —CHR₁— is selected from —CH₂— and (R) —CH(CH₂)—. It is particularly preferred that said moieties Y, Z, Ar, R₂, R₃ and R₆ are embodied as set forth above.

In this second aspect of the present invention, it is most preferred that the compound is selected from a group consisting of:
(R)-4-[(3-cyclohexylpropyl)-(2-phenylpropionyl)amino]-butyramide (1);
4-[(3-cyclohexylpropyl)-(2-naphthalen-2-yl-acetyl)amino]butyramide (8); and
8-[(3-cyclohexylpropyl)-(2-naphthalen-2-yl-acetyl)amino]octanamide (9).

The number in parenthesis denotes the compound as referred to in the following.

The present inventive use is typically practised via a pharmaceutical composition comprising a compound as set forth above as active ingredient in association with a pharmaceutically acceptable adjuvant, diluent or carrier.

The pharmaceutical composition may be adapted for oral, intravenous, topical, intraperitoneal, nasal, buccal, sublingual or subcutaneous administration or for administration via the respiratory tract e.g. in the form of an aerosol or an air-suspended fine powder. The composition may thus for instance be in the form of tablets, capsules, powders, microparticles, granules, syrups, suspensions, solutions, transdermal patches or suppositories.

It should be noted that the composition used herein may optionally include two or more of the above outlined compounds.

The pharmaceutical composition may optionally comprise e.g. at least one further additive selected from a disintegrating agent, binder, lubricant, flavoring agent, preservative, colorant and any mixture thereof. Examples of such and other additives are found in “Handbook of Pharmaceutical Excipients”; Ed. A. H. Kibbe, 3^{rd} Ed., American Pharmaceutical Association, USA and Pharmaceutical Press UK, 2000.

The pharmaceutical composition in a solid dosage form is typically a perorally available tablet. A tablet may be manufactured by compression of a suitable granulate by procedures well established in the art. Examples of suitable tablet compressing equipment are rotary presses provided by Elizabeth-Hata International, USA, and Courtoy NV, BE. For a comprehensive overview of pharmaceutical tablet manufacturing, see “Tableting” (by N. A. Armstrong) in “Pharmaceutics—The science of dosage form design”, pp 647-668; Ed. M. E. Aulton, Churchill Livingstone, Edinburgh, London, Melbourne and New York, 1988.

In another embodiment the invention relates to a method for treatment of IBD, wherein said method comprises administering to an animal, including human, patient of a therapeutically effective amount of a compound as outlined above. Treatment of Crohn's disease and ulcerative colitis, alone or in combination, is especially preferred.

The typical dosage of the compounds used according to the present invention varies within a wide range and will depend on various factors such as the individual requirements of each patient and the route of administration. The dosage is generally within the range of 0.01-100 mg/kg body weight. A medical practitioner of ordinary skill in the art will be able to optimise the dosage to the circumstances at hand.

### EXPERIMENTAL

### General

The present compounds where X is the radical —NH— and both R₂ and R₃ are H, i.e. hydrogen, can be prepared by solid phase synthesis in accordance with the following general synthetic scheme (Scheme 1). Y is O with the exemplified reagents used in the reaction steps i-viii. Z and R₆ are selected among the previously defined alternatives. The ball symbol used in the schemes herein is a conventional representation of a resin. It may typically be a TentaGel Rink Amide Resin.

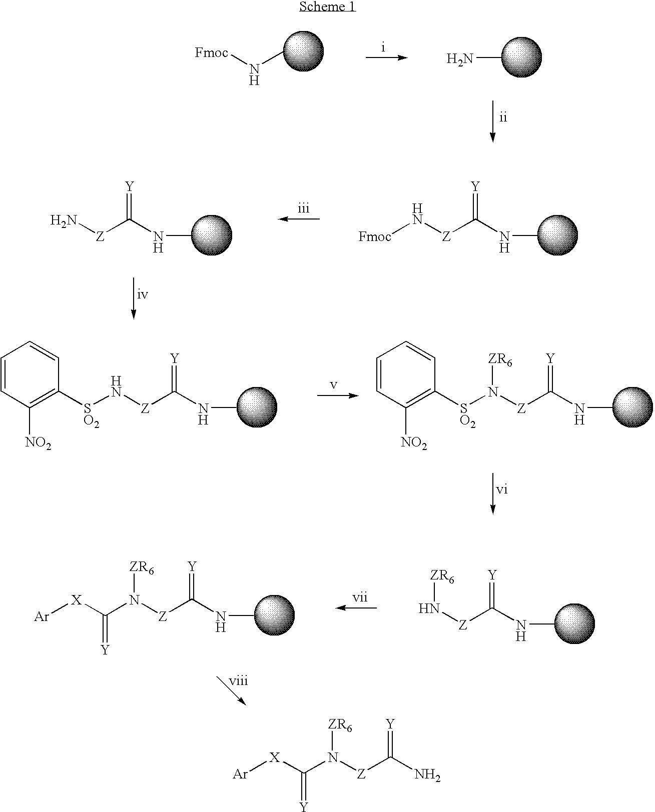

Examples of the above reagents are: i) 25% PIP/DMF; ii) Fmoc-NH—Z—CO₂H/HOBt/DIC, 3 eq; iii) 25% PIP/DMF; iv) o-NBS-Cl, 4 eq, collidine, 6 eq; v) R₆Z—OH/TPP/DIAD, 10 eq; vi) HSCH₂CH₂OH/DBU/DMF, 10 eq; vii) ArNCO, 10 eq; viii) TFA/H₂O/TIS, 96/2/2.

The following abbreviations are used:

Abu aminobutyric acid residue

BAL backbone amide linker

Boc tert-butyloxycarbonyl

DBU 1,8-diazabicyclo[5.4.0]undec-7-ene

DCM dichloromethane

DIAD diisopropyl azodicarboxylate

DIC 1,3-diisopropyl carbodiimide

DIPEA N,N-diisopropylethylamine

DME 1,2-dimethoxyethane

DMF N,N-dimethylformamide

DMSO dimethyl sulphoxide

DSS dextran sodium sulphate

eq equivalent

Fmoc 9-fluorenylmethyloxycarbonyl

h hour

HOBt 1-hydroxybenzotriazole

HPLC high performance liquid chromatography

MeOH methanol

min minutes

Mp melting point

MS mass spectrometry

o-NBS-Cl o-nitrobenzenesulfonyl chloride

Ph phenyl

PIP piperidine

rt room temperature

TFA trifluoroacetic acid

TIS triisopropylsilane

TNBS 2,4,6-trinitrobenzene sulphonic acid

TPP triphenylphosphine

To obtain N-monoalkylated amides, i.e. where R₂≠H and R₃=H, the synthesis is performed on Rink amide resin protected with the o-NBS group (o-NBS-TentaGel-S-RAM resin). The resin is alkylated with R₂OH/TPP/DIAD under Mitsunobu reaction conditions. The o-NBS group is subsequently removed with a 2-mercaptoethanol/DBU/DMF cocktail. Alternatively, backbone amide linker (BAL) resin is reductively aminated with R₂NH₂. In both cases the resin bound secondary amine is subsequently acylated with Fmoc-NH—Z—CO₂H/DIC. The following standard reference literature provides further guidance on general experimental set up, as well as on the availability of required starting material and reagents, in the particular steps utilised in providing the compounds used in the present invention:

Fukuyama, T.; Jow, C.-K.; Cheung, M. “2- and 4-Nitrobenzenesulfonamides: Exceptionally Versatile Means for Preparation of Secondary Amines and Protection of Amines” Tetrahedron Lett. 36:6373-6374 (1995);

Mitsunobu, O. “The Use of Diethyl Azodicarboxylate and Triphenylphosphine in Synthesis and Transformation of Natural Products” Synthesis 1-28 (1981);

Miller, S. C.; Scanlan, T. S. “Site-Selective N-Methylation on Solid Support” J. Am. Chem. Soc. 119:2301-2302 (1997);

Jensen, K. J.; Alsina, J.; Songster, M. F.; Vagner, J.; Albericio, F.; Baranay, G. “Backbone Amide Linker Strategy for Solid-Phase Synthesis of C-Terminal-Modified and Cyclic Peptides” J. Am. Chem. Soc. 120:5441-5452 (1998); and

Rink, Hans “Solid-phase synthesis of protected peptide fragments using a trialkoxy-diphenyl-methyl ester resin” Tetrahedron Lett. 28:3787-3790 (1987).

The following specific examples shall not be construed as a limitation of how the invention may be practised.

### Experimental

### Synthesis

### Synthesis of Original Library (Cf. Steps i & ii)

Unless otherwise provided, all synthetic steps were performed at room temperature.

14 portions of Fmoc-TentaGel-S-RAM resin (0.25 mmol/g, 2.75 g each; provided from Rapp Polymere, Tübingen, DE) were treated with a 25% solution of PIP in DMF for 30 min. The resins were thoroughly washed with DMF (2×), MeOH (2×) and DMF (2×) and subsequently acylated with 14 different Fmoc-ω-amino acids using DIC/HOBt coupling methodology with a 3-fold excess of reagents. The progress of the reaction was monitored with a conventional Kaiser's ninhydrine test.

Introduction of the o-NBS Group (Cf. Steps iii & iv):

The Fmoc groups were removed by treatment with 25% PIP in DMF for 30 min and the resins were thoroughly washed as above, followed by treatment with a solution of o-NBS-Cl (4 eq) and collidine (6 eq) in DCM for 1 h. The progress of the reaction was monitored with a conventional Kaiser's ninhydrine test. Upon reaction completion the resins were suspended in DMSO/CHCl₃ (4:1) and the suspensions were combined. The resulting slurry was split into 16 portions (about 0.6 mmol each) and the resins were placed in 16 manual solid phase synthesis vessels and washed with dry DME.

Mitsunobu Alkylation (Cf. Step v):

Each resin was then suspended in 6 ml of a solution containing 20 eq of an alcohol in dry DME. Selection criteria for the alcohols were based on results in the optimisation phase of the synthesis to provide good diversity and yields in the alkylation reactions. To each resin was then added 20 eq of a preformed TPP/DIAD complex dissolved in dry DME, and the reaction was carried out overnight. Aliquots of all 16 resins were cleaved with TFA and analysed by HPLC (Waters 600 Chromatograph) and MS (Finningan MAT Spectrometer).

Removal of the o-NBS Group (Cf. Step vi):

The resins were treated with 20 eq of 1 M solutions of 2-mercaptoethanol and DBU in DMF for 1.5 h, and were washed thoroughly after the completion of the reaction.

Final Acylations (Cf. Step vii):

The resins were transferred to 8 reaction blocks each having 96 wells. Each resin was then split into 48 portions and placed in the same row of blocks 1 to 4 or 5 to 8, respectively. The blocks were then arranged as shown below:

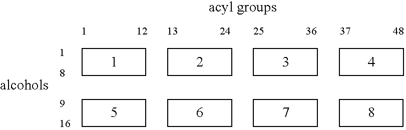

Columns 1 to 4 were acylated with Fmoc-amino acids, columns 5 to 20 were acylated with Boc-amino acids, and columns 21-37 with carboxylic acids using 15 eq of an acid and 15 eq of DIC. Column 38 was treated with 15 eq of acetic anhydride/DIPEA. For the synthesis of the sulphonamides (columns 39-43) an appropriate sulphonyl chloride (15 eq) and DIPEA (22.5 eq) were used. Finally the ureas (columns 44-48) were formed using an appropriate isocyanate (15 eq) in DMF. All the reactions were carried out overnight at rt. The completion of the reactions was confirmed (one test per column) with a conventional chloranil test. The Fmoc groups (columns 1 to 4) were then removed by treatment with 25% PIP/DMF, after which the entire library was washed out with DMF, MeOH and DCM followed by drying in vacuo.

Cleavage (Cf. Step Viii):

The compounds were cleaved from the resin by treatment with 0.5% H₂O in TFA overnight. The resin was removed by filtration and the filtrates were collected in 8 plates each having 96 wells. The solvent was removed by evaporation in a conventional Savanth centrifuge. The library was reconstituted in MeOH/H₂O (1:1, v/v), and the solvents were evaporated in a Savanth centrifuge. The final product was an original library consisting of 768 mixtures each containing 14 compounds.

Synthesis of Deconvolution Library:

By screening of the original library against hCB2-R, 12 active wells were identified. Since each well contained 14 compounds the deconvolution library needed to consist of 168 discrete compounds. The deconvolution library and two subsequent optimisation libraries were obtained according to the protocol for the original library, albeit the mix and split steps were omitted.

### Synthesis of 4-[1-(3-cyclohexylpropyl)-3-phenylureido]butyramide (compound 17)

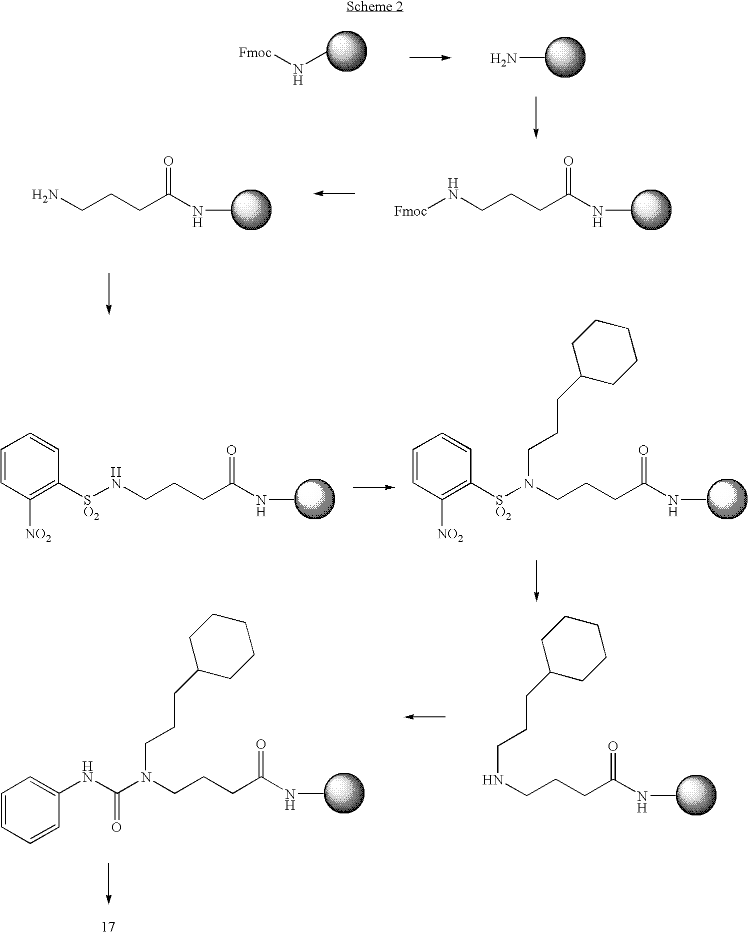

10 g of Fmoc-TentaGel-S-RAM resin (0.25 mmol/g, 2.5 mmol) was treated with 25% PIP in DMF for 30 min. The resin was washed with DMF (2×), MeOH (2×) and DMF (2×) and subsequently acylated with Fmoc-γ-Abu-OH/DIC/HOBt (3 eq) in DMF. The completeness of the reaction was assessed with Kaiser's ninhydrine test. The Fmoc group was removed followed by resin washing as described above. The o-NBS group was introduced by treatment with o-NBS-Cl (4 eq)/collidine (6 eq) in DCM for 1 h at rt. The resin was then suspended in dry DME (15 ml) and 3-cyclohexyl-1-propanol (3.8 ml, 25 mmol, 10 eq) was added. The TPP/DIAD complex was preformed at 0° C. by dissolving TPP (6.55 g, 25 mmol, 10 eq) in dry DME (30 ml) and adding DIAD (4.92 ml, 25 mmol, 10 eq). The complex was then added to the suspension and the reaction was carried out overnight. An aliquot of the resin was cleaved and analysed by HPLC (column: Vydac C18, 5μ, 250×4.6 mm; solvents: A-0.1% TFA (aq), B-80% CH₃CN/0.1% TFA (aq); a linear gradient of B was used). The content of the non-alkylated substrate was below 2%. The o-NBS group was subsequently removed by treatment with 1 M 2-mercaptoethanol/DBU in DMF (25 ml) for 1 h (2×). The resin was then treated with PhNCO (10.9 ml, 25 mmol, 10 eq) in DMF for 4 h. The completeness of the reaction was confirmed by a negative chloranil test. The compound was cleaved from the resin by treatment with TFA/TIS/H₂O 96/2/2 (100 ml) for 1.5 h at rt. The resin was filtered off and the solvents were evaporated. The crude product was purified by preparative HPLC. The fractions containing the pure compound were combined and lyophilised. The obtained product was treated with isopropyl ether, whereby crystalline compound was provided. Yield: 442.8 mg (51%, 1.28 mmol); Mp. 104-106° C.; MS (ion spray): [M+H]⁺ expected 346.2, observed 346.2; ¹H NMR (500 MHz, CDCl₃) data was consistent with the structure of compound 17.

### Experimental

### Biology

### Example 1

### compound 17 in 2,4,6-trinitrobenzene sulphonic acid (TNBS) Colitis Induction Model

Animals were housed ten per cage and had free access to standard mouse chow and tap water. For colitis induction, C57Bl/6 mice were anesthetized for 90-120 minutes and received an intra-rectal administration of TNBS (40 μL, 150 mg/kg; provided by Sigma-Aldrich, FR) dissolved in a 1:1 mixture of 0.9% NaCl with 100% ethanol. Control mice received a 1:1 mixture of 0.9% NaCl with 100% ethanol or a saline solution using the same technique. Animals were sacrificed 5 days after TNBS administration. The anti-inflammatory effects of compound 17 were tested by administering the compound once daily by subcutaneous injection, starting one day before colitis induction. Macroscopic, histology and biologic assessments of colitis were performed blindly by two investigators.

The colon of each mouse was examined under a dissecting microscope (magnification, ×5) to evaluate the macroscopic lesions according to the established so-called Wallace criteria. The Wallace score rates macroscopic lesions on a scale from 0 to 10 based on features reflecting inflammation, such as hyperemia, thickening of the bowel, and extent of ulceration (Wallace, J. L. et al. Gastroenterology 96(1):29-36 (1989)). A colon specimen located 2 cm above the anal canal was cut into three parts, one of which was fixed in 4% paraformaldehyde and embedded in paraffin. Sections stained with May-Grunwald-Giemsa were examined blindly by two investigators and scored according to the so-called Ameho criteria (Ameho, C. K. et al. Gut 41(4):487-493 (1997)). This grading on a scale from 0 to 6 takes into account the degree of inflammation infiltrate, the presence of erosion, ulceration, or necrosis, and the depth and surface extension of lesions.

Control mice sacrificed five days after administration of 50% ethanol or a saline solution showed no macroscopic or histologic lesions in the colon. Mice receiving TNBS injections had severe lesions five days after colitis induction, showing necrosis of the colon and leading to mortality in 70% of the mice. TNBS-induced colitis was characterized by hyperemia and extensive area of ulceration. These lesions were characterized by neutrophilic infiltration extending to the mucosa deep into the muscular layer.

To test the ability of compound 17 to prevent or protect against TNBS-induced colitis, compound 17 was administered subcutaneously preventively one day before colitis induction, and then once daily until animal sacrifice. Five days after TNBS administration, treatment with compound 17 (10⁻¹ mg/kg/d) resulted in a decrease in Wallace score (3.8±0.89 vs 7.0±0.37) and Ameho score (3.9±0.62 vs 5.3±0.15) compared to untreated mice with TNBS-induced colitis. Improvement of histologic lesions was characterized by a reduction of the number of neutrophils in the lamina propria and an inflammation limited to the mucosa without ulceration. This dose was not associated with a significant improvement of mortality rates.

### Example 2

### Compound 17 in Dextran Sulfate Sodium (DSS) Colitis Induction Model

Mice were fed 5% DSS (molecular weight 30-40 kDa) for seven days. DSS was dissolved in sterile distilled water and given ad libitum throughout the experiment.

Stool consistency, occult blood or the presence of macroscopic rectal bleeding gross were determined daily and blindly by two experiment investigators, as described previously (Hartmann et al., J Pharmacol Exp Ther., 292(1):22-30 (2000)). Briefly, for stool consistency, 0 point was given for well formed pellets, 2 points for pasty and semi-formed stools that did not stick to the anus, and 4 points for liquid stools that remained adherent to the anus. Bleeding was scored 0 point for no blood in hemoccult, 2 points for positive hemoccult, and 4 points for gross bleeding from the rectum. These scores were added, resulting in a total clinical score ranging from 0 (healthy) to 8 (maximal activity of colitis). Post mortem, rings of the trans-verse part of the colon were fixed in 4% formaldehyde and embedded in paraffin for histologic analysis. Sections (4 μm) were stained with May-Grunwald-Giemsa and histologic scoring performed. For cell infiltration of inflammatory cells, rare inflammatory cells in the lamina propria were counted as 0; increased numbers of inflammatory cells, including neutrophils in the lamina propria as 1; confluence of inflammatory cells, extending into the submucosa as 2; and a score of 3 was given for transmural extension of the inflammatory cell infiltrate. For epithelial damage, absence of mucosal damage was counted as 0, discrete focal lymphoepithelial lesions were counted as 1, mucosal erosion/ulceration was counted as 2, and a score of 3 was given for extensive mucosal damage and extension through deeper structures of the bowel wall. The two sub-scores were added and the combined histologic score ranged from 0 (no changes) to 6 (extensive cell infiltration and tissue damage).

Seven day oral administration of DSS resulted in acute colitis characterized from the 5^{th} day by anal bleeding, weight loss and diarrhea. Histologically, lesions were associated with severe epithelial damages and an inflammatory infiltrate extending deeply from the mucosa into the muscular layer. Preventive treatment with compound 17 at the dose of 10⁻¹ mg/kg/d significantly reduced scores for clinical parameters (2.8±0.13 vs 3.7±0.14; P=0.003) and histologic score (3.9±0.39 vs 5.8±0.13; P=0.001). The main clinical parameters modified by the tested compound were blood levels and stool consistency. Histological evaluation of treated mice was characterized by a mild neutrophil infiltration limited to the mucosa and a preservation of epithelium integrity.

### Example 3

### hCB2-R Binding for Compounds 1-17

The assay used was essentially set up as described in Munro, S., Thomas, K. L., Abu-Shaar, M. in “Molecular characterisation of a peripheral receptor for cannabinoids” Nature 365:61-65 (1993).

It deserves mentioning that to date two different cannabinoid receptors have been cloned from mammalian tissues, and these are denoted CB1 and CB2. The central and most of the peripheral effects of cannabinoids are the result of CB1 activation. This receptor is abundant in the central nervous system where it mediates cannabinoid psychoactivity. CB1 is also present in peripheral nerve terminals and in non-neuronal sites, such as the testis, uterus, eyes, vascular endothelium and immune cells. CB2 is predominantly present in peripheral tissues that are associated with immune functions, i.e. spleen, tonsils, B-cells and macrophages, whereas it is not detectable in neurons.

Monoclonal HEK cell lines with stable expression of the human CB1 receptor and CHO-K1 cell lines with stable expression of hCB2-R were established. The CB binding assays were performed with membranes prepared from these cell lines. The CB2 ligand binding mixture contains 0.3-0.5 nM [³H]-CP55940, 7 μg of CB2 membranes and the test compounds in a concentration range of from 1.0×10⁻⁴ to 1.0×10⁻¹² M. The assay buffer comprises 50 mM Tris-HCl (pH 7.4), 5 mM MgCl₂, 2.5 mM EDTA and 5 mg/ml fatty acid-free bovine serum albumin. The binding mixtures are incubated for 2 h at 30° C. and terminated by rapid filtration (Brandel 96 well cell harvester) over 934AH filters (Whatman) followed by 6 washes with ice-cold binding buffer. The filters are dried and [³H]-CP55940 bound radioactivity is determined by liquid scintillation counting. Non-specific binding is determined in the presence of 10 μM CP55940. The binding data is analysed with the program GraphPad Prism (provided by GraphPad Software, San Diego, Calif., USA). The Kᵢ values presented in table 1 were obtained.

TABLE 1
Binding constants (Kᵢ) to hCB2-R.

| Compound | Kᵢ (μM) |
|---|---|
| 1 | 2.9 |
| 2 | 0.45 |
| 3 | 0.12 |
| 4 | 0.78 |
| 5 | 1.1 |
| 6 | 2.2 |
| 7 | 1.5 |
| 8 | 2.7 |
| 9 | 15 |
| 10 | 1 |
| 11 | 0.77 |
| 12 | 0.41 |
| 13 | 0.45 |
| 14 | 0.84 |
| 15 | 0.21 |
| 16 | 0.13 |
| 17 | 0.11 |

Increased expression of CB2 positive mast cells in diseased gastrointestinal human tissue, viz the muscle layer, compared to normal colon was also observed. It is speculated that the common hCB2-R binding property is responsible for the anti-IBD efficacy of the present compounds.

The biological results as set forth above establish that the compounds of formula (I) are suitable for the treatment of IBD, including Crohn's disease and ulcerative colitis.

All of the literature referred to is to be regarded as an integral part of the present writ.

1. A method of treating inflammatory bowel disease comprising administering to an animal in need thereof a compound having the formula (I): 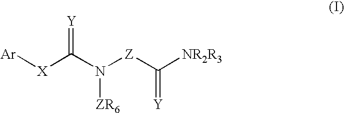
wherein
X is selected from —NR₁— and —CHR₁—;
Y is independently selected from O and S;
Z is independently selected from a C₁₋₇ straight or C₄₋₈ branched alkylene chain, a C₂₋₇ alkenylene chain, and a part of a C₃₋₈ cycloalkyl or C₅₋₈ cykloalkenyl ring structure;
Ar is an aryl group selected from aromatic carbocyclic ring systems, five- or six-membered heteroaromatic ring systems and bicyclic heteroaromatic ring systems;
R₁, R₂ and R₃ are independently selected from substituents (a)-(d):
(a) H;
(b) C₁₋₆ straight or C₄₋₈ branched chain alkyl;
(c) C₃₋₈ cycloalkyl or C₅₋₈ cykloalkenyl; and
(d) C₂₋₆ alkenyl or alkynyl;
wherein substituents (b)-(d) optionally have at least one substituent independently selected from (e)-(i):
(e) Ar, O—Ar or S—Ar;
(OH, O-alkyl or S-alkyl, where alkyl is selected from substituents (b)-(c);
(g) NR₄R₅, where R₄ and R₅ are independently selected from substituents (a)-(d), or optionally together form a nitrogen containing ring structure comprising from 2 to 5 carbon atoms;
(h) NH—C(O)-alkyl, C(O)-alkyl, O—C(O)-alkyl or S—C(O)-alkyl, where alkyl is selected from substituents (b)-(c); and
(i) F, Cl or Br;
R₆ is selected from the group consisting of Ar and substituents (a)-(c), where (b) and (c) are optionally substituted with at least one of substituents (e)-(i); and
Ar optionally has at least one substituent independently selected from substituents (b)-(i); or
tautomers, solvates and pharmaceutically acceptable salts of said compound.

2. The method according to claim 1, wherein said inflammatory bowel disease is Crohn's disease or ulcerative colitis.

3. The method according to claim 1, wherein X is —NR₁—.

4. The method according to claim 3, wherein R₁ is H.

5. The method according to claim 1, wherein Y is O.

6. The method according to claim 1, wherein Ar is selected from phenyl and naphthyl.

7. The method according to claim 1, wherein Z is selected from —CH₂—, —(CH₂)₂—, —(CH₂)₃—, —(CH₂)₅—, —(CH₂)₆—, —(CH₂)₇— and trans-2-cyclohexylene.

8. The method according to claim 1, wherein R₆ is selected from isopropyl, cyclopentyl, cyclohexyl, phenyl, 4-n-butylphenyl, 4-isopropylphenyl and 2-naphthyl.

9. The method according to claim 1, wherein R₂ and R₃ are independently selected from H and 4-chlorobenzyl.

10. The method according to claim 1, wherein the compound is selected from the group consisting of:
4-[3-phenyl-1-(6-phenylhexyl)ureido]butyramide;
4-[1-(4-butylbenzyl)-3-phenylureido]butyramide;
4-[1-(4-isopropylbenzyl)-3-phenylureido]butyramide;
4-[1-(4-methylpentyl)-3-phenylureido]butyramide;
N-(4-chlorobenzyl)-4-[1-(3-cyclohexylpropyl)-3-phenylureido]butyramide;
trans-2-[1-(3-cyclohexylpropyl)-3-phenylureido]cyclo-hexanecarboxamide;
4-[1-(3-cyclohexylpropyl)-3-naphthalen-2-yl-ureido]butyramide;
4-[1-(2-naphthalen-2-yl-ethyl)-3-phenylureido]-butyramide;
4-[1-(2-cyclohexylethyl)-3-phenylureido]butyramide;
4-(1-phenethyl-3-phenylureido)butyramide;
4-(1-benzyl-3-phenylureido)butyramide;
4-[1-(3-cyclopentylpropyl)-3-phenylureido]butyramide;
4-[3-phenyl-1-(5-phenylpentyl)ureido]butyramide; and
4-[1-(3-cyclohexylpropyl)-3-phenylureido]butyramide.

11. The method according to claim 1, wherein X is —CHR₁—.

12. The method according to claim 11, wherein said radical —CHR₁— is selected from —CH₂— and (R)—CH(CH₃)—.

13. The method according to claim 11, wherein Y is O, Z is selected from —CH₂—, —(CH₂)₂—, —(CH₂)₃—, —(CH₂)₅—, —(CH₂)₆—, —(CH₂)₇— and trans-2-cyclohexylene, Ar is selected from phenyl and naphthyl, R₂ and R₃ are independently selected from H and 4-chlorobenzyl, and R₆ is selected from isopropyl, cyclopentyl, cyclohexyl, phenyl, 4-n-butylphenyl, 4-isopropylphenyl and 2-naphthyl.

14. The method according to claim 11, wherein the compound is selected from a group consisting of:
(R)-4-[(3-cyclohexylpropyl)-(2-phenylpropionyl)amino]butyramide;
4-[(3-cyclohexylpropyl)-(2-naphthalen-2-yl-acetyl)amino]butyramide; and
8-[(3-cyclohexylpropyl)-(2-naphthalen-2-yl-acetyl)amino]octanamide.

15. The method according to claim 1, wherein the animal is a human.

16. (canceled)

17. The method according to claim 1, comprising administering a dosage of said compound of 0.01-100 mg/kg body weight of the animal.

18. The method according to claim 1, wherein said compound, or tautomer, solvate or pharmaceutically acceptable salt thereof, is administered as a composition further comprising a pharmaceutically acceptable adjuvant, diluent, or carrier.

19. The method according to claim 18, wherein said composition is adapted for oral, intravenous, topical, intraperitoneal, nasal, buccal, sublingual or subcutaneous administration, or for administration via the respiratory tract.

20. The method according to claim 18, wherein said composition is in the form of a tablet, capsule, powder, microparticle, granule, syrup, suspension, solution, transdermal patch, suppository, aerosol or air-suspended fine powder.

21. The method according to claim 18, wherein said composition comprises two or more compounds of formula (I), or tautomers, solvates or pharmaceutically acceptable salts thereof.
